(19) United States
(12) Patent Application Publication
(10) Pub. No.: US 2019/0117800 A1
(43) Pub. Date: Apr. 25, 2019

(54) SYSTEMS AND METHODS FOR FILLING VIALS WITH GASES

(71) Applicant: Microvascuar Therapeutics LLC, Tucson, AZ (US)

(72) Inventors: Evan C. Unger, Tucson, AZ (US); Daniel C. Evans, Tucson, AZ (US); Varadarajan Ramaswami, Tucson, AZ (US)

(21) Appl. No.: 16/093,617

(22) PCT Filed: Apr. 19, 2017
(86) PCT No.: PCT/US17/28257

§ 371 Date: Oct. 12, 2018

Related U.S. Application Data
(60) Provisional application No. 62/324,599, filed on Apr. 19, 2016.

(51) Int. Cl. A61K 49/22; B65B 3/00

(52) U.S. Cl. CPC A61K 49/223; A61J 1/18; B65B 3/003; A61K 49/225

(57) ABSTRACT

The invention provides novel apparatus and methods for efficiently and accurately filling vials with gaseous materials such as a fluorinated gas, with or without concomitantly filling the vials with another liquid or solid material.

### PRIORITY CLAIMS AND RELATED PATENT APPLICATIONS

This application claims the benefit of priority from U.S. Provisional Application Ser. No. 62/324,599, filed on Apr. 19, 2017, the entire content of which is incorporated herein by reference in its entirety.

### TECHNICAL FIELDS OF THE INVENTION

This invention generally relates to systems and methods for deploying gaseous materials. More particularly, the invention relates to apparatus and methods for efficiently and accurately filling vials with gaseous materials, such as a fluorinated gas, with or without concomitantly filling the vials with another liquid or solid material.

### BACKGROUND OF THE INVENTION

Fluorinated gases have found many uses in the medical field, for example, to make ultrasound contrast agents. Definity®, comprised of phospholipid-coated perfluoropropane microbubbles, is an example of one such product. Definity® is manufactured by first preparing phospholipid suspension, which is aseptically filled into sterile lyophilization vials followed by exchange of headspace air above the liquid suspension for perfluoropropane gas using a lyophilization chamber. The final step of this manufacturing process is done by placing the vials containing the phospholipid suspension inside the lyophilization chamber, cooling and evacuating the atmosphere inside the chamber, and then releasing the octafluoropropane gas into the chamber. The vials, after they have been filled with gas, are stoppered, and the remaining gas inside the chamber (>>90%) is reclaimed.

Such a method of filling gas is expensive due to the high cost of perfluoropropane gas ($1,940/kg) in addition to the cost associated with the use of lyophilizer itself. Additionally, perfluoropropane gas is heavier than air and may distribute unevenly in the lyophilization chamber, which can result in variations in the concentration of gas in the different vials due to their different positions within the lyophilization chamber.

Thus, an ongoing need remains for a simpler, more efficient and economical system and method that greatly reduces waste of the fluorinated gas and ensures accuracy, reproducibility and uniformity of products.

### SUMMARY OF THE INVENTION

The present invention is based in part on the discovery of novel apparatus and methods that allow efficiently and accurately filling of vials with gaseous materials, such as a fluorinated gas, with or without concomitantly filling the vials with another liquid or solid material.

In one aspect, the invention generally relates to a method for filling vials with a gaseous material. The method includes: injecting the gaseous material into the headspace of a vial; and sealing the vial to securely contain the gaseous material inside the vial. In certain embodiments, the method further includes, prior to or simultaneous with injecting the gaseous material into the vial, filling the vial with a liquid material. In certain embodiments, the method includes evacuating the vial headspace prior to filling of the liquid and or/gaseous material.

In another aspect, the invention generally relates to a vial pre-filled with a fluorinated gaseous material according to a method according to a method disclosed herein.

In yet another aspect, the invention generally relates to a filling system for simultaneously filling a vial with a gaseous material and a liquid material, The system includes: a combined filling nozzle comprising a gas purging nozzle and a liquid filling nozzle; a sleeve attached to the filling nozzle providing a seal on the vial being filled; a pneumatic solenoid valve; one or more peristaltic pumps; a decapper/capper; and a control system.

### BRIEF DESCRIPTION OF THE DRAWINGS

The invention will be better understood from a reading of the following detailed description taken in conjunction with the drawings in which like reference designators are used to designate like elements, and in which:

FIG. 1 illustrates an experimental embodiment of a setup of the present invention.

FIG. 2 illustrates an exemplary embodiment of a combined liquid/gas-filling nozzle.

FIG. 3 illustrates an exemplary embodiment of a gas fill following vial headspace evacuation.

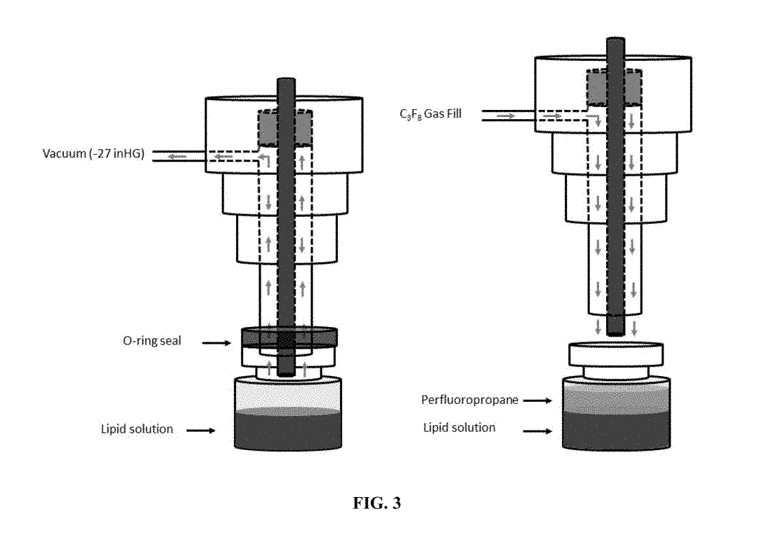

FIG. 4 illustrates an exemplary embodiment of a filling system.

FIG. 5 illustrates an exemplary embodiment of a filling system.

FIG. 6 illustrates an exemplary embodiment of an array of multiple nozzles allowing multiple vials to be filled in parallel.

### DETAILED DESCRIPTION OF THE INVENTION

The invention provides a filling system and related methods that deliver a gaseous material (e.g., a fluorinated gas) directly into pre-filled vials. The invention may be used to fill vials containing or being concurrently filled with aqueous suspensions of biological materials such as lipids, proteins or other film forming materials. The invention may be used to fill vials containing or being concurrently filled with dried powdery materials (e.g., dried lipids with materials such as polyethyleneglycol) or dried microspheres comprised of materials such as polylactic acid or polylactide-co-glycolide.

In one aspect, the invention generally relates to a method for filling vials with a gaseous material. The method includes: injecting the gaseous material into the headspace of a vial; and sealing the vial to securely contain the gaseous material inside the vial. In certain embodiments, the method further includes, prior to or simultaneous with injecting the gaseous material into the vial, filling the vial with a liquid material.

In certain preferred embodiments, filling the vial with a liquid material is performed prior to injecting the gaseous material into the vial and injecting the gaseous material into the headspace of a vial is to fill the headspace above the liquid material.

In certain preferred embodiments, filling the vial with a liquid material is performed simultaneous with injecting the gaseous material into the vial.

In certain preferred embodiments, evacuating the vial headspace is performed after the liquid fill and before injecting gaseous material into the vial.

The liquid material can be an aqueous suspension, which may include one or more compounds or agents. In certain embodiments, the aqueous suspension comprises lipids (e.g., dipalmitoylphosphatidylcholine (DPPC), dipalmitoylphosphatidylethanolamine (DPPE), dipalmitoylphosphatidylethanolamine-monomethoxy-PEG(5,000) (DPPE-MPEG-5000). Examples of preferred lipids include phospholipids 1,2-dipalmitoyl-sn-glycero-3-phosphocholine, 16:0 PC (DPPC), 1,2-dimyristoyl-sn-glycero-3-phosphocholine, 14:0 PC (DMPC), 1,2-distearoyl-sn-glycero-3-phosphocholine, 18:0 PC (DSPC), 1,2-dipalmitoyl-sn-glycero-3-phosphoethanolamine, 16:0 PE, 1,2-dilauroyl-sn-glycero-3-phosphoethanolamine, 12:0 PE, 1,2-dipentadecanoyl-sn-glycero-3-phosphoethanolamine, 15:0 PE, 1,2-distearoyl-sn-glycero-3-phosphoethanolamine, 18:0 PE, 1,2-dimyristoyl-sn-glycero-3-phosphoethanolamine, 14:0 PE, 1,2-distearoyl-sn-glycero-3-phosphoethanolamine-N-[methoxy(polyethylene glycol)-5000] (ammonium salt), 18:0 PEG5000 PE, 1,2-dipalmitoyl-sn-glycero-3-phosphoethanolamine-N-[methoxy(polyethylene glycol)-5000] (ammonium salt), 16:0 PEG5000, 1,2-dimyristoyl-sn-glycero-3-phosphoethanolamine-N-[methoxy(polyethylene glycol)-5000] (ammonium salt), 14:0 PEG5000 PE, 1,2-dipalmitoyl-sn-glycero-3-phosphoethanolamine-N-[methoxy(polyethylene glycol)-3000] (ammonium salt), 16:0 PEG3000 PE, 1,2-dimyristoyl-sn-glycero-3-phosphoethanolamine-N-[methoxy(polyethylene glycol)-5000] (ammonium salt), 14:0 PEG5000 PE, 1,2-dipalmitoyl-sn-glycero-3-phosphoethanolamine-N-[methoxy(polyethylene glycol)-20000] (ammonium salt), 16:0 PEG2000 PE.

Other examples of lipids include monogalactosyldiacylglycerol (MGDG), monoglucosyldiacylglycerol (MGDG), diphosphatidylglycerol (DPG) also called cardiolipin, phosphatidylserine (PS), phosphatidylethanolamine (PE) and diacylglycerol. Phosphatidic acid (PA) is also a cone-shaped lipid, but is not preferred due to its propensity to hydrolysis and potential to cause bioeffects. The most preferred cone-shaped phospholipid is phoshatidylethanolamine (PE). Cone shaped lipids comprises a head group that occupies a smaller volume than do the pendent groups extending outwardly from head group (e.g., phosphatidylethanolamine). Cylindrical-shaped lipid comprises a head group that occupies a similar volume as that volume defined by the pendent groups extending outwardly from head group (e.g., phosphatidylcholine). In addition, the applicants cationic, i.e. positively charged lipids can be used as cone shaped lipids provided that the head group of said cationic lipid is smaller than the tail. Examples of potentially useful cone-shaped cationic lipids include but are not limited to 1,2-dioleoyl-3-trimethylammonium-propane (chloride salt), 1,2-dioleoyl-3-trimethylammonium-propane (methyl sulfate salt), 1,2-dimyristoyl-3-trimethylammonium-propane (chloride salt), 1,2-dipalmitoyl-3-trimethylammonium-propane (chloride salt), 1,2-distearoyl-3-trimethylammonium-propane (chloride salt), 1,2-dioleoyl-3-dimethylammonium-propane, 1,2-dimyristoyl-3-dimethylammonium-propane, 1,2-dipalmitoyl-3-dimethylammonium-propane, 1,2-distearoyl-3-dimethylammonium-propane, dimethyldioctadecylammonium and 1,2-di-O-octadecenyl-3-trimethylammonium propane (chloride salt), O,O-di-O-octadecenyl-3-tα-trimethylammonioacetyl-diethanolamine. Microbubbles prepared with a third lipid—a cone-shaped lipid, in particular DPPE, provide better bubble count and better microbubble stability than formulations without such a third lipid. Preferably the cone-shaped lipid is provided within the formulation at a concentration of between about 5 and about 20 mole percent and more preferably at about 8 to 15 mole percent and most preferably at about 10% of the total lipid in the formulation.

A fourth lipid, a bifunctional PEG'ylated lipid may be employed. Bifunctional PEG'ylated lipids include but are not limited to DSPE-PEG(2000) Succinyl 1,2-distearoyl-sn-glycero-3-phosphoethanolamine-N-[succinyl(polyethylene glycol)-2000] (ammonium salt), DSPE-PEG(2000) PDP 1,2-distearoly-sn-glycero-3-phosphoethanolamine-N-[PDP(polyethylene glycol)-2000] (ammonium salt), DSPE-PEG(2000) Maleimide 1,2-distearoly-sn-glycero-3-phosphoethanolamine-N-[maleimide(polyethylene glycol)-2000] (ammonium salt), DSPE-PEG(2000) Biotin 1,2-distearoyl-sn-glycero-3-phosphoethanolamine-N-[maleimide(polyethylene glycol)-2000] (ammonium salt), DSPE-PEG(2000) Cyanur 1,2-distearoly-sn-glycero-3-phosphoethanolamine-N-[cyanur(polyethylene glycol)-20000] (ammonium salt), DSPE-PEG(2000) Amine 1,2-distearoyl;-sn-glycero-3-phosphoethanolamine-N-[amino(polyethylene glycol)-2000] (ammonium salt), DPPE-PEG(5,000)-maleimide, 1,2-distearoyl-sn-glycero-3-phosphoethanolamine-N-[dibenzocyclooctyl(polyethylene glycol)-20000] (ammonium salt), 1,2-distearoyl-sn-glycero-3-phosphoethanolamine-N-[azido(polyethylene glycol)-2000] (ammonium salt), 1,2-distearoyl-sn-glycero-3-phosphoethanolamine-N-[succinyl(polyethylene glycol)-2000] (ammonium salt), 1,2-distearoyl-sn-glycero-3-phosphoethanolamine-N-[carboxy(polyethylene glycol)-2000] (ammonium salt), 1,2-distearoyl-sn-glycero-3-phosphoethanolamine-N-[maleimide(polyethylene glycol)-2000] (ammonium salt), 1,2-distearoyl-sn-glycero-3-phosphoethanolamine-N-[PDP(polyethylene glycol)-2000] (ammonium salt), 1,2-distearoyl-sn-glycero-3-phosphoethanolamine-N-[amino(polyethylene glycol)-2000] (ammonium salt), 1,2-distearoyl-sn-glycero-3-phosphoethanolamine-N-[biotinyl(polyethylene glycol)-2000] (ammonium salt), 1,2-distearoyl-sn-glycero-3-phosphoethanolamine-N-[cyanur(polyethylene glycol)-2000] (ammonium salt), 1,2-distearoyl-sn-glycero-3-phosphoethanolamine-N-[folate(polyethylene glycol)-2000] (ammonium salt), 1,2-distearoyl-sn-glycero-3-phosphoethanolamine-N-[folate(polyethylene glycol)-5000] (ammonium salt), N-palmitoyl-sphingosine-1-{succinyl[methoxy(polyethylene glycol)2000]} and N-palmitoyl-sphingosine-1-{succinyl[methoxy(polyethylene glycol)5000]}.

The bifunctional lipids may be used for attaching antibodies, peptides, vitamins, glycopeptides and other targeting ligands to the microbubbles. The PEG chains MW may vary from about 1,000 to about 5,000 Daltons in the third lipid. In certain embodiments, the PEG chain MW are from about 2,000 to about 5,000 Daltons.

The lipid chains of the lipids used in the invention may vary from about 14 to about 20 carbons in length. Most preferably the chain lengths are from about 16 to about 18 carbons. Chains may be saturated or unsaturated but are preferably saturated. Cholesterol and cholesterol derivatives may also be employed in the invention with the proviso that they be neutral, or if negatively charged contain a head group greater than about 350 MW in juxtaposition to the negative charge to shield the charge from the biological milieu.

In certain preferred embodiments, filling the vial with a powdery or lyophilized material is performed prior to injecting the gaseous material into the vial.

The powdery or lyophilized material may be any suitable material, for example, all classes of phospholipids, sugars, or sugar alcohols.

Any suitable gaseous material may be any suitable gaseous material. In certain preferred embodiments, the gaseous material comprises a fluorinated gas. The term “fluorinated gas”, as used herein, refers to hydrofluorocarbons, which contain hydrogen, fluorine and carbons, and compounds containing sulfur and fluorine. In the context of the present invention the term refers to materials that are comprised of carbon and fluorine or sulfur and fluorine in their molecular structure and are gases at normal temperature and pressure.

In certain preferred embodiments, the gaseous material comprises a non-fluorinated gas. Examples of non-fluorinated gases include oxygen (O₂), air, nitrogen (N₂), nitrous oxide (N₂O), nitric oxide (NO), ozone (O₃). When the non-fluorinated gas is included in the vial, it generally constitutes from 10-90 mole percent of the gases in the vial and the fluorinated gas comprises between 90-10 mole percent of the gases. More preferably, the non-fluorinated gas comprises from about 5 to about 15 mole percent of the gases and the fluorinated gas comprises the remainder. More than on non-fluorinated gas may be included in the vial and more than on type of fluorinated gas.

Table 1 shows exemplary gases useful in the invention. The preferred gases have molecular weights ranging from about 146 to about 338 and boiling points ranging from about about −64° C. to about 56.6° C. Preferred gases include sulfur hexafluoride, perfluoropropane, perfluorobutane, perfluoropentane and perfluorohexane. For the highest molecular weight gases, these can be volatilized by heating to temperatures above their respective boiling points. Preferably the fluorinated gas has 80% or higher concentration in the vial after it is sealed. The resulting vials can be activated to produce microbubbles by mechanical agitation, e.g., with a VialMix by shaking the sealed vials at 4,500 rpm for 45 seconds.

TABLE 1
Examples of different gases useful in the invention

| | | Aqueous Solubility | |
|---|---|---|---|
| | Molecular | (Ostwald's | Boiling Point |
| Compound | Weight | Coefficient) | (° C.) |
| Nitrogen | 28 | 18071 | −196 |
| Oxygen | 32 | 4865 | −183 |
| Sulfur | 146 | 5950 | −64 |
| Hexafluoride | | | |
| Perfluoropropane | 188 | 583 | −36.7 |
| Perfluorobutane | 238 | <500 | −1.7 |
| Perfluoropentane | 288 | >24 and <500 | 29 |
| Perfluorohexane | 338 | 24 | 56.6 |

In certain preferred embodiments, the fluorinated gas includes a material selected from the group consisting of sulfur hexafluoride, perfluoropropane, perfluorobutane, perfluoropentane and perfluorohexane. In certain preferred embodiments, the fluorinated gas comprises perfluoropropane. In certain preferred embodiments, the fluorinated gas consists of perfluoropropane.

In another aspect, the invention generally relates to a vial pre-filled with a fluorinated gaseous material according to a method disclosed herein.

The pre-filled vials have total volumes of from about 1.0 to about 5.0 mL. More preferably the vials are from about 2.0 to about 3.0 total volume. Preferably the liquid phase ranges from about 30 to about 75% of the total volume. In certain embodiments, the pre-filled vial is filled with about 1.0 to 2.0 mL of a fluorinated gas (e.g., perfluoropropane). In certain preferred embodiments, the pre-filled vial is filled with from about 4 mg to about 16 mg (e.g., from about 4 mg to about 12 mg, from about 4 mg to about 10 mg, from about 4 mg to about 8 mg, from about 6 mg to about 16 mg, from about 8 mg to about 16 mg, from about 10 mg to about 16 mg, about 4 mg, 5 mg, 6 mg, 7 mg, 8 mg, 9 mg, 10 mg, 12 mg, 14 mg, 16 mg) of a fluorinated gas (e.g., perfluoropropane). In certain preferred embodiments, the pre-filled vial is further filled with about 0.5 mg to about 5.0 mg (e.g., about 0.5 mg to about 3.0 mg, about 0.5 mg to about 2.0 mg, about 0.5 mg to about 1.0 mg, about 1.0 mg to about 5.0 mg, about 2.0 mg to about 5.0 mg, about 3.0 mg to about 5.0 mg) of lipids.

In yet another aspect, the invention generally relates to a filling system for simultaneously filling a vial with a gaseous material and a liquid material, The system includes: a combined filling nozzle comprising a gas purging nozzle and a liquid filling nozzle; a sleeve attached to the filling nozzle providing a seal on the vial being filled; a pneumatic solenoid valve; one or more peristaltic pumps; and a decapper/capper.

In certain embodiments, the system further includes a control system for controlling the filling operation.

In certain embodiments, the system further includes: a first container for holding a gaseous material; a second container for holding a liquid material; one or more regulator valves for regulating the first and second containers; and one or more flow meters for measuring the flow rate of the gaseous material and the liquid material.

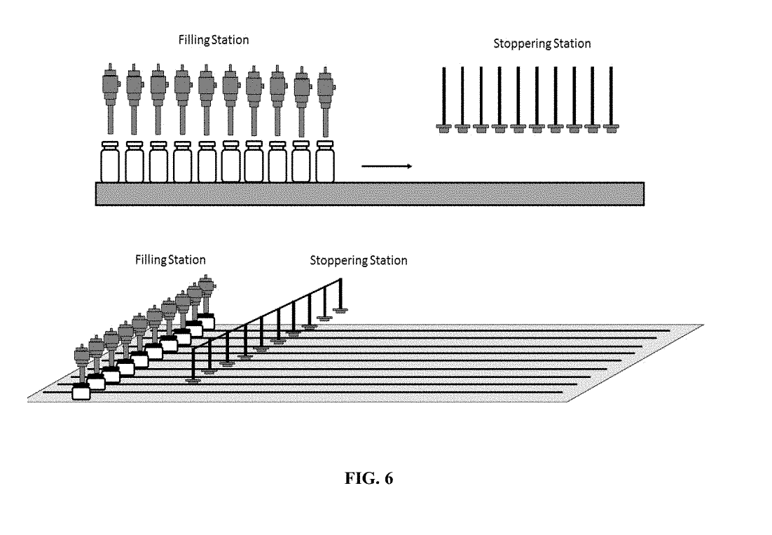

In certain embodiments, the combined filling nozzle comprising an outer gas purging nozzle and an inner liquid filling nozzle. In certain embodiments, the combined filling nozzle comprising an outer gas purging nozzle and an inner liquid filling nozzle is provided in an array of multiple nozzles allowing multiple vials to be filled in parallel (see, e.g., FIG. 6).

### EXAMPLES

A blend of lipids is prepared containing dipalmitoylphosphatidylcholine (DPPC), dipalmitoylphosphatidylethanolamine (DPPE) and dipalmitoylphosphatidylethanolamine-monomethoxy-PEG(5,000) (DPPE-MPEG-5000). Each mL of the resultant lipid blend contained 0.75 mg total lipid (consisting of 0.400 mg DPPC, 0.046 mg DPPE, and 0.32 mg DPPE-MPEG-5000). Each mL of the lipid blend also contained 103.5 mg propylene glycol, 126.2 mg glycerin, 234 mg sodium phosphate monobasic monohydrate, 2.16 mg sodium phosphate dibasic heptahydrate, and 4.87 mg sodium chloride in Water for Injection. The pH was 6.2-6.8. The materials were provided in 2.0 mL Wheaton (VWR 223683) lyophilization vials—1.5 mL fill volume. These materials were used in the feasibility studies described herein.

### Example 1

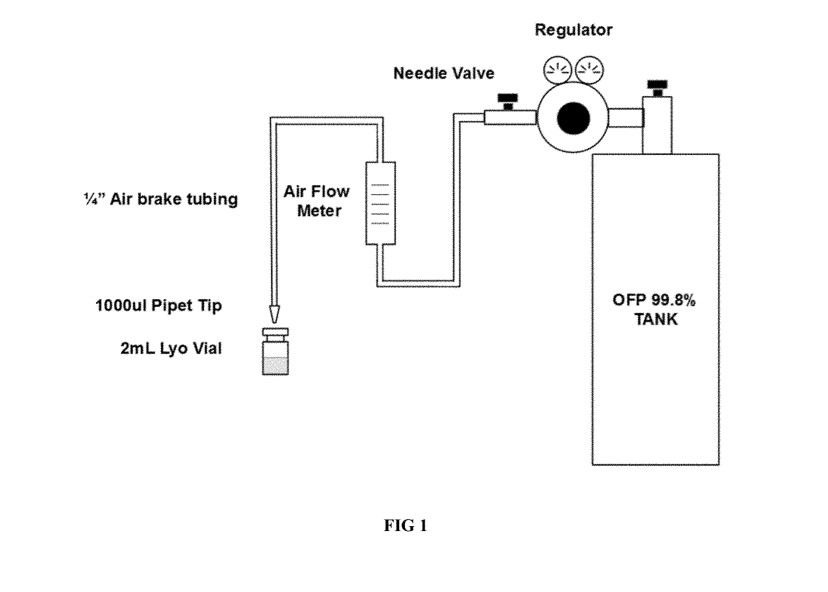

The following experimental setup, illustrated in FIG. 1, was assembled to determine the feasibility of filling vials containing the MVT-100 phospholipid suspension with octafluoropropane gas without the use of a lyophilization chamber. A Western Medica (MSH15973) gas regulator was attached to a gas cylinder containing 99.8% Octafluoropropane provided by FluoroMed (APF-N40M). Parker-Hannafin air brake tubing (PFT-4A-BLK-1000) was connected to the regulator needle valve and an air flow meter (Gilmont No. 12) using a Parker quick connect fittings (W68PLP-4-4). The downstream side of the flow meter was connected to a VWR 1000 μL pipet tip (82028-568) using air brake tubing. The pipet tip was placed just above a Wheaton (VWR 223683) lyophilization vial with the tip partially in the vial. The regulator was set to 5 PSI and the needle valve was set for 2 different flow rates (5 and 10m1/sec) through the pipet tip. Vials were filled with gas for 3 different time lengths (at 2, 5, and 10 seconds, timed with a cell phone stopwatch) immediately after which a Wheaton (VWR W224100-093) stopper and aluminum crimp seal (VWR 16171-819) were placed on the vial. Vials were then sampled for OFP concentration using gas chromatography. This procedure was meant to simulate the filling of individual vials via the gas purge line that exists on many sterile manufacturing filling systems. The results of this study are listed in Table 2.

TABLE 2

| GAS FLOW | | | | |
|---|---|---|---|---|
| RATE | GAS FILL TIME | % OFP | AVG | STD DEV |
| 5 mL/sec | 2 sec | 90.24 | 90.60 | 2.64 |
| 5 mL/sec | 2 sec | 88.15 | | |
| 5 mL/sec | 2 sec | 93.40 | | |
| 5 mL/sec | 5 sec | 89.64 | 92.85 | 3.04 |
| 5 mL/sec | 5 sec | 93.22 | | |
| 5 mL/sec | 5 sec | 95.68 | | |
| 5 mL/sec | 10 sec | 94.35 | 94.58 | 2.34 |
| 5 mL/sec | 10 sec | 92.37 | | |
| 5 mL/sec | 10 sec | 97.03 | | |
| 10 mL/sec | 2 sec | 90.87 | 92.15 | 1.15 |
| 10 mL/sec | 2 sec | 92.50 | | |
| 10 mL/sec | 2 sec | 93.08 | | |
| 10 mL/sec | 5 sec | 92.85 | 91.78 | 1.23 |
| 10 mL/sec | 5 sec | 90.44 | | |
| 10 mL/sec | 5 sec | 92.06 | | |
| 10 mL/sec | 10 sec | 87.65 | 90.32 | 2.38 |
| 10 mL/sec | 10 sec | 90.69 | | |
| 10 mL/sec | 10 sec | 92.35 | | |

### Example 2

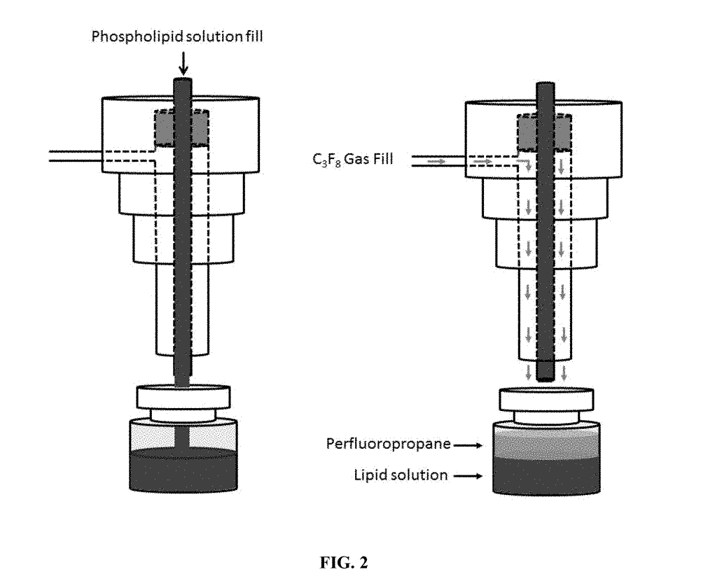

A Flexicon peristaltic pump (PD12I), controller (MC12), and combination filling (30-040-016)/gas purging (30-031-050) nozzle (FIG. 2) along with an SMC pneumatic solenoid valve (VCL21-5D-3-02N-H-Q) and a combination flow meter/control valve (Dwyer RMA-3-SSV) were used to further investigate filling vials of MVT-100 with octafluoropropane immediately after the liquid fill. 1.6 mm sterile tubing was used to connect the reservoir containing bulk MVT-100 phospholipid with the liquid filling nozzle via the peristaltic pump. Silicone tubing from a gas cylinder containing 99.8% octafluoropropane (FluoroMed—APF-N40M) was connected to the gas-purging nozzle with a flow meter/control valve and the pneumatic solenoid valve in between to control the rate and duration of gas flow. The controller was set to fill a 2 mL Wheaton serum vial (VWR 223683) with 1.5 mL of the phospholipid suspension while a second microcontroller (Atmel Atmega 328P) and relay (Songle SRD-05V-SL-C) were used to actuate the solenoid valve immediately after to fill the vial with OFP gas. The peristaltic pump speed was set to deliver the liquid at 200 RPM, while the regulator pressure and flowmeter/controller were set at 10 PSI and 2.0 SCFH (15.73 mL/sec) respectively to deliver the gas for 2, 3, and 4 seconds, immediately after the gas filling was complete the vial was sealed with a rubber stopper (VWR W224100-093) and aluminum crimp seal (VWR 16171-819). The results of these experiments are shown in Table 3.

TABLE 3

| GAS FLOW | GAS FILL | | | |
|---|---|---|---|---|
| RATE | TIME | % OFP | AVG | ST DEV |
| 15.73 mL/sec | 2.0 sec | 89.97 | 90.23 | 0.75 |
| 15.73 mL/sec | 2.0 sec | 89.64 | | |
| 15.73 mL/sec | 2.0 sec | 91.08 | | |
| 15.73 mL/sec | 3.0 sec | 90.84 | 92.14 | 1.19 |
| 15.73 mL/sec | 3.0 sec | 92.43 | | |
| 15.73 mL/sec | 3.0 sec | 93.16 | | |
| 15.73 mL/sec | 4.0 sec | 91.54 | 92.91 | 1.22 |
| 15.73 mL/sec | 4.0 sec | 93.30 | | |
| 15.73 mL/sec | 4.0 sec | 93.89 | | |

### Example 3

In the second set of experiments the gas fill was started 1 second prior to and during the liquid fill. Table 4 below data for the samples filled with gas before and during the liquid fill.

TABLE 4

| GAS FLOW | GAS FILL | | | |
|---|---|---|---|---|
| RATE | TIME | % OFP | AVG | STDEV |
| 15.73 mL/sec | 3.0 sec | 88.52 | 88.74 | 3.05 |
| 15.73 mL/sec | 3.0 sec | 91.90 | | |
| 15.73 mL/sec | 3.0 sec | 85.81 | | |
| 15.73 mL/sec | 4.0 sec | 94.85 | 94.87 | 0.07 |
| 15.73 mL/sec | 4.0 sec | 94.81 | | |
| 15.73 mL/sec | 4.0 sec | 94.95 | | |
| 15.73 mL/sec | 5.0 sec | 95.53 | 95.80 | 0.37 |
| 15.73 mL/sec | 5.0 sec | 95.64 | | |
| 15.73 mL/sec | 5.0 sec | 96.22 | | |

The above set of experiments show that sequential filling of the vials with liquid followed by gas yields a higher and more consistent concentration of perfluoropropane at 3 second total gas filling time, however at 4 seconds the samples gas prior to and during the liquid fill have a slightly higher octafluoropropane concentration.

### Example 4

For the 3rd set of experiments filling of octafluoropropane gas was done using flow rates of 1.57 mL/sec, 2.36 mL/sec, 3.15 mL/sec, and 3.93 mL/sec. The results of the filling vials containing MVT-100 at lower gas flow rates are shown in Table 5 below.

TABLE 5

| GAS FLOW | GAS FILL | | | |
|---|---|---|---|---|
| RATE | TIME | % OFP | AVG | STDEV |
| 1.57 mL/s | 2 Second | 85.39 | 86.59 | 3.44 |
| 1.57 mL/s | 2 Second | 83.92 | | |
| 1.57 mL/s | 2 Second | 90.47 | | |
| 2.36 mL/s | 2 Second | 83.16 | 85.25 | 2.42 |
| 2.36 mL/s | 2 Second | 87.91 | | |
| 2.36 mL/s | 2 Second | 84.67 | | |
| 3.15 mL/s | 2 Second | 86.53 | 86.59 | 1.04 |
| 3.15 mL/s | 2 Second | 87.66 | | |
| 3.15 mL/s | 2 Second | 85.59 | | |
| 3.93 mL/s | 1 Second | 82.63 | 83.11 | 1.23 |
| 3.93 mL/s | 1 Second | 84.51 | | |
| 3.93 mL/s | 1 Second | 82.19 | | |
| 3.93 mL/s | 2 Second | 84.92 | 87.28 | 2.07 |
| 3.93 mL/s | 2 Second | 88.10 | | |
| 3.93 mL/s | 2 Second | 88.81 | | |

### Example 5

A blend of lipids is prepared containing DPPC, DPPE and DPPE-MPEG-5000 Each mL of the resultant lipid blend contains 0.75 mg total lipid (consisting of 0.400 mg DPPC, 0.046 mg DPPE, and 0.32 mg MPEG-5000-DPPE) and 1.5 mg of PEG(5,000). Each mL of the lipid blend also contains 103.5 mg propylene glycol, 15 mg of PEG(5,000), 2.34 mg sodium phosphate monobasic monohydrate, 2.16 mg sodium phosphate dibasic heptahydrate, and 4.87 mg sodium chloride in Water for Injection. The pH is 6.2-6.8. The materials are provided in 3.0 mL Wheaton (VWR 223683) lyophilization vials—1.5 mL fill volume. The vials are then lyophilized yielding a cake of white powder in each vial. The Flexicon system is used to fill the vials with perfluoropropane gas (1.0 PSI, 1.0 second fill) and the vials are sealed resulting in a lyophilized powder of lipids, PEG(5,000) and other excipients. The microbubbles are reconstituted by injecting WFI into the vials (optionally using a vent needle to avoid increasing the pressure in the vials). The vial is gently agitated by hand and the microbubbles are withdrawn into a syringe. The product is an injectable suspension of microbubbles useful for ultrasound imaging.

### Example 6

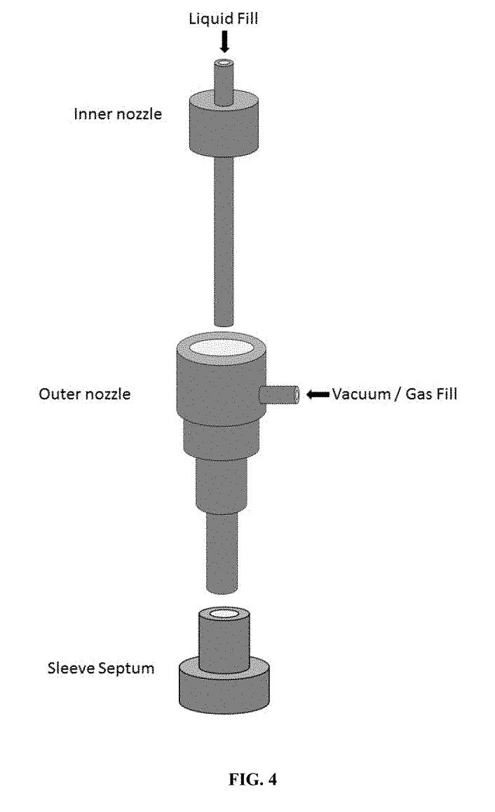

A Flexicon FF20 automatic vial handling system connected to a PD12I peristaltic pumps, MC12I controller, and UP20 decapper/capper was used to determine if filling the headspace of sterile vials with a perfluorocarbon gas using a commercially available sterile filling system was possible. This on-line filling of perflurocarbon gas was done using the combination of gas purging nozzle (Flexicon 30-031-050) and liquid filling nozzle (Flexicon 30-040-010) illustrated in FIG. 4.

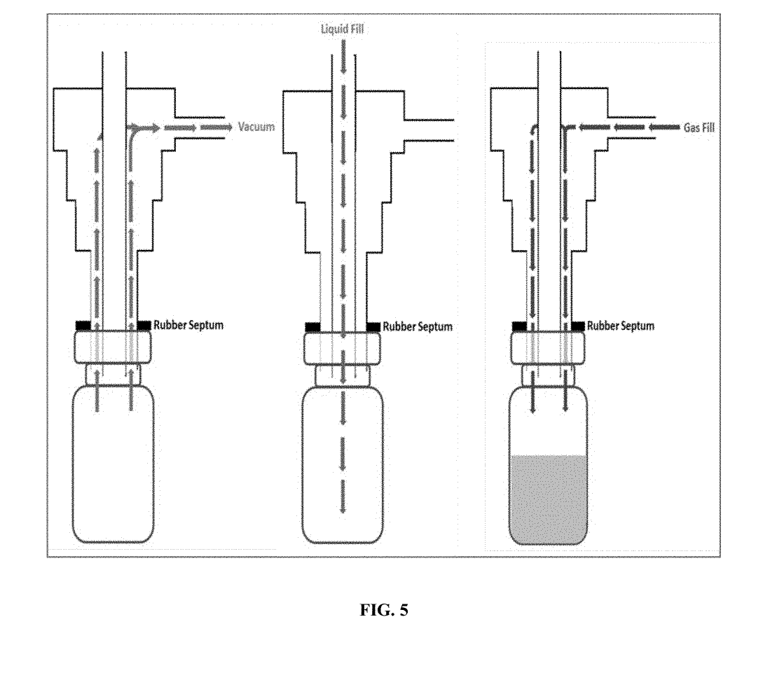

Once the sterile vial (2mL Wheaton 223683) was moved into the fill position of the FF20, the vial's rubber septum was removed by the UP20 and the combination filling nozzle was moved into the filling position. In this filling position, a size 13-sleeve stopper (Fisher Scientific 14-126AA) attached to the filling nozzle provided a seal on the vial. Once the filling nozzle was in position a pneumatic solenoid valve allowed vacuum to be pulled on the vial via a KNF UN820.3FTP vacuum pump. After a preset vacuum time, the vial was then filled simultaneously with both liquid (phospholipid suspension) and a perfluorocarbon gas (using an additional pneumatic solenoid valve) for a preset time. Both solenoid valves were actuated in sequence using a 24-volt signal sent from the filling system. Once the liquid and gas fill were complete, the vial stopper was placed back on the vial by the UP20 and a new vial was moved into position. An illustration of this filling sequence is shown in FIG. 5.

Gas filling data for both perfluoropropane (C3F8) and perfluorobutane are shown in Table 6. 10 vials were filled using the method listed above after which GC analysis was done to determine the percentage of gas in the headspace. During the filling of the gas, the regulator was set to 10 PSI and the flow rate was controlled using a flow meter with control valve (Dwyer RMA-6-SSV). The vacuum duration for perfluoropropane and perfluorobutane were 0.5 and 1 second respectively. The gas flow rate and flow duration for perfluoropropane was 8.38mL/sec for 1 second while the flow rate and duration for perfluorobutane was 16.34 mL/sec for 2 seconds.

TABLE 6

| | Perfluoropropane | Perfluorobutane |
|---|---|---|
| SAMPLE # | % C₃F₈ | % C₄F₁₀ |
| 01 | 89.89 | 95.94 |
| 02 | 90.60 | 93.96 |
| 03 | 91.12 | 94.96 |
| 04 | 86.66 | 90.58 |
| 05 | 87.19 | 93.57 |
| 06 | 91.54 | 86.63 |
| 07 | 89.94 | 92.68 |
| 08 | 92.71 | 94.41 |
| 09 | 90.05 | 95.14 |
| 10 | 87.51 | 98.72 |
| Average = | 89.72 | 93.66 |
| Std Dev = | 1.99 | 3.25 |
| Rel Std = | 2.22% | 3.47% |

An additional study was done to determine if filling with gas without pulling a vacuum would provide high enough concentrations of perfluoropropane in the vial headspace. Table 7 shows the results of this study for 2 different gas-liquid fill sequences. Both sequences filled with gas for 3 seconds at 1.97 mL/sec (measured by Dwyer RMA-3-SSV flow meter). Sequence 1 started the liquid suspension fill 1.0 seconds after the start of the gas fill while Sequence 2 started the liquid fill 1.5 seconds after the start of the gas fill.

TABLE 7

% PERFLUOROPROPANE

| SAMPLE # | SEQUENCE 1 | SEQUENCE 2 |
|---|---|---|
| 01 | 90.89 | 87.71 |
| 02 | 89.62 | 89.34 |
| 03 | 90.17 | 90.79 |
| 04 | 89.73 | 88.98 |
| 05 | 92.41 | 91.44 |
| 06 | 91.13 | 87.95 |
| AVG = | 90.66 | 89.37 |
| STD = | 1.05 | 1.50 |
| RSD = | 1.16% | 1.68% |

Data from the above and other experiments showed that the vacuum step is not necessary; however, it does decrease the total process time by 1.5 seconds/vial. The vacuum step is therefore useful as it enables a higher throughput of filling of vials with the filling line.

### Example 7

In a vial containing a phospholipid suspension, dodecafluoropentane (DDFP) gas is filled in the headspace using as described in Example 6 above for perfluoropropane and perfluorobutane. Evaporation of DDFP is done by heating the liquid above its boiling point (29° C.) in a sealed container thereby saturating the space above the liquid with DDFP gas. Heated lines connecting the vessel containing DDFP gas with the vial-filling nozzle is used to prevent condensation of DDFP during the vial headspace fill. A solenoid valve is used to actuate the gas fill when the vial was in position. Either pulling vacuum on the vial headspace or a carrier gas such as nitrogen is used to create the driving force necessary to move the DDFP gas into the vial headspace.

As one skilled in the art would recognize, it may be necessary to heat the filling lines to above the boiling point of the fluorocarbon when employing DDFP or perfluorohexane. Heated lines (e.g. >29° C., the boiling point of DDFP) connecting the vessel containing DDFP gas with the vial filling nozzle can be used to prevent condensation of DDFP during the vial headspace fill. A solenoid valve can be used to actuate the gas fill when the vial was in position. Either pulling vacuum on the vial headspace or a carrier gas such as nitrogen could be used to create the driving force necessary to move the DDFP gas into the vial headspace.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance that can be adapted to the practice of this invention in its various embodiments and equivalents thereof.

What is claimed is:

1. A method for filling vials with a gaseous material, comprising:
injecting the gaseous material into the headspace of a vial; and
sealing the vial to securely contain the gaseous material inside the vial.

2. The method of claim 1, further comprising, prior to or simultaneous with injecting the gaseous material into the vial,
filling the vial with a liquid material.

3. The method of claim 2, wherein filling the vial with a liquid material is performed prior to injecting the gaseous material into the vial.

4. The method of claim 2, wherein filling the vial with a liquid material is performed simultaneous with injecting the gaseous material into the vial.

5. The method of claim 2, wherein the liquid material is an aqueous suspension.

6. The method of claim 5, wherein the aqueous suspension comprises lipids.

7. The method of claim 1, further comprising, prior to or simultaneous with injecting the gaseous material into the vial,
filling the vial with a powdery or lyophilized material.

8. The method of claim 7, wherein filling the vial with a powdery or lyophilized material is performed prior to injecting the gaseous material into the vial.

9. The method of any of claims 1-8, wherein the gaseous material comprises a fluorinated gas.

10. The method of any of claims 1-8, wherein the gaseous material comprises a non-fluorinated gas.

11. The method of claim 9, wherein the fluorinated gas comprises a material selected from the group consisting of sulfur hexafluoride, perfluoropropane, perfluorobutane, perfluoropentane and perfluorohexane.

12. The method of claim 11, wherein the fluorinated gas comprises perfluoropropane.

13. The method of claim 11, wherein the fluorinated gas is perfluoropropane.

14. A vial pre-filled with a fluorinated gaseous material according to a method according to any of claims 1-9 and 11-13.

15. The pre-filled vial of claim 14, wherein the fluorinated gaseous material is perfluoropropane.

16. The pre-filled vial of claim 15, comprising between about 4 mg/mL and about 8 mg/mL of perfluoropropane.

17. The pre-filled vial of claim 15, comprising between amount 0.3 mg/mL and 5 mg/mL of lipids.

18. The pre-filled vial of claim 15, comprising between about 4 mg/mL and about 8 mg/mL of perfluoropropane and between amount 0.3 mg/mL and 5 mg/mL of lipids.

19. A filling system for simultaneously filling a vial with a gaseous material and a liquid material, comprising:
a combined filling nozzle comprising a gas purging nozzle and a liquid filling nozzle;
a sleeve attached to the filling nozzle providing a seal on the vial being filled;
a pneumatic solenoid valve;
one or more peristaltic pumps; and
a decapper/capper.

20. The filling system of claim 19, further comprising a control system for controlling the filling operation.

21. The filling system of claim 19 or 20, further comprising:
a first container for holding a gaseous material;
a second container for holding a liquid material;
one or more regulator valves for regulating the first and second containers; and
one or more flow meters for measuring the flow rate of the gaseous material and the liquid material.

22. The filling system of claim 19-21, wherein the combined filling nozzle comprising an outer gas purging nozzle and an inner liquid filling nozzle.

23. The filling system of any of claims 19-22, wherein the combined filling nozzle comprising an outer gas purging nozzle and an inner liquid filling nozzle is provided in an array of multiple nozzles allowing multiple vials to be filled in parallel.
